(19) United States
(12) Patent Application Publication
(10) Pub. No.: US 2020/0230073 A1
(43) Pub. Date: Jul. 23, 2020

(54) NON-AQUEOUS PATCH COMPRISING LIDOCAINE

(71) Applicants: ITOCHU CHEMICAL FRONTIER Corporation, Tokyo (JP); OISHI KOSEIDO CO., LTD., Saga (JP)

(72) Inventors: Tatsuya Mori, Tosu-shi (JP); Naoyuki Saida, Tosu-shi (JP)

(21) Appl. No.: 16/588,766

(22) Filed: Sep. 30, 2019

Related U.S. Application Data
(63) Continuation of application No. 15/904,071, filed on Feb. 23, 2018, now abandoned.
Application No. 15/904,071 is a continuation of application No. PCT/JP2016/075376, filed on Aug. 24, 2016.
(60) Provisional application No. 62/208,985, filed on Aug. 24, 2015.

(51) Int. Cl. A61K 9/70; A61K 9/00; A61K 31/167; A61P 23/00; A61K 47/10; A61K 47/12

(52) U.S. Cl. CPC A61K 9/7053; A61K 9/0014; A61K 47/12; A61P 23/00; A61K 47/10; A61K 31/167

(57) ABSTRACT

The present invention relates to non-aqueous patches containing lidocaine.

### CROSS-REFERENCE TO RELATED APPLICATIONS

This application is a continuation of U.S. patent application Ser. No. 15/904,071, filed on Feb. 23, 2018, which is a continuation of International Application No. PCT/JP2016/075376, filed Aug. 24, 2016, which claims priority to and the benefit of U.S. Provisional Patent Application No. 62/208,985, filed Aug. 24, 2015, the subject matter of all of which are herein incorporated by reference in their entirety.

### FIELD OF THE INVENTION

The present invention relates to non-aqueous patches containing lidocaine.

### BACKGROUND OF THE INVENTION

Lidocaine is used for the purpose of local anesthesia or topical anesthesia. The usage form of lidocaine is an external preparation comprising lidocaine or a patch comprising lidocaine. Examples of external preparations include ointment, cream, jelly, spray, etc., which are used, for example, for topical anesthesia of the skin in the treatment of postherpetic neuralgia. Examples of patches include aqueous base patches (cataplasms) and non-aqueous patches (tapes).

An example of aqueous base patches is Lidoderm® which is mainly used for topical anesthesia of the skin in the treatment of postherpetic neuralgia, and is also used to relieve muscle pain. Many aqueous base patches have thick plasters because they contain moisture; therefore, aqueous base patches are poorly compatible with the skin and thus are difficult to attach to the skin for long durations. Furthermore, the vaporization of moisture from the patch causes changes in adhesion and physical properties. Additionally, in order to make lidocaine permeate the muscle, it is necessary to dissolve lidocaine, and moisture is thus required to dissolve lidocaine.

Patent Japanese Patent No. 3159688 discloses a technique for alleviating postherpetic neuralgia, in which 5 to 30 wt. % of lidocaine is added as a local anesthetic. Japanese Unexamined Patent Publication No. 7-215850 discloses a technique relating to a percutaneous absorption tape for local anesthesia comprising 5 to 100 wt. % of lidocaine. Japanese Unexamined Patent Publication No. 9-315964 and Japanese Unexamined Patent Publication No. 2001-392501 disclose techniques relating to a patch comprising 0.5 to 5 wt % of lidocaine. These patent publications suggest using a small amount of lidocaine, and can be used for household use; however, even after the small amount of lidocaine is completely dissolved, the lidocaine cannot be stably released over a long period of time (e.g., 12 hours or more) and cannot permeate into the skin. Thus, there is a problem with the pain-relieving effect of the patches as described.

WO 2009/060629 discloses a technique relating to a patch comprising 10 to 40 wt % of lidocaine. These non-aqueous patches have poor permeability to the skin because the lidocaine is not dissolved and is present in a crystalline state. In addition, the technique disclosed therein uses a high concentration of lidocaine. Lidocaine has an adverse effect on the heart. Prolonged use of a high concentration of lidocaine causes side effects, such as shock, rubor, and irritating sensation. External preparations comprising more than 5 wt % of lidocaine are designated as powerful drugs, and cannot be used as household (nonprescription) medicine. Moreover, aqueous based lidocaine containing preparations have poor adhesive properties and thus these patches fall off easily. In addition, while lidocaine dissolve easily in organic solvents such as methanol, ethanol, diethyl ether, and the like, it is difficult to dissolve in water and thus lidocaine is not completely dissolved in aqueous patches.

### SUMMARY OF THE INVENTION

The present invention relates to non-aqueous tapes and patches containing lidocaine and methods of administering these tapes and patches so that patients receive an effective amount of lidocaine without causing undue side effects.

The present invention relates to non-aqueous tapes and patches that contain less lidocaine but are bioequivalent to aqueous lidocaine patches. The present invention relates to non-aqueous tapes and patches that contain less lidocaine than aqueous patches but have one or more pharmacokinetic parameters of the formulation is within 70% to 125% of that of an aqueous patch containing 5% lidocaine.

The present invention relates to methods for treating pain in a patient by administering to the patient a lidocaine tape which has about 1.8 to about 5.6 wt % lidocaine such that one or more pharmacokinetic parameters of the formulation is within 70% to 125% of that of an aqueous patch containing 5% lidocaine.

The present invention relates to methods for treating pain in a patient administering to the patient a lidocaine tape comprising about 1.0%-5.6 wt % lidocaine and about 10%-50% terpene.

The present invention relates to methods for treating postherpetic neuralgia in a patient by administering to the patient a lidocaine tape comprising 1.8 wt % lidocaine.

### FIGURES

FIG. 1—Graphic comparison of the mean blood concentration of Lidoderm® and LIDT-185 over time.

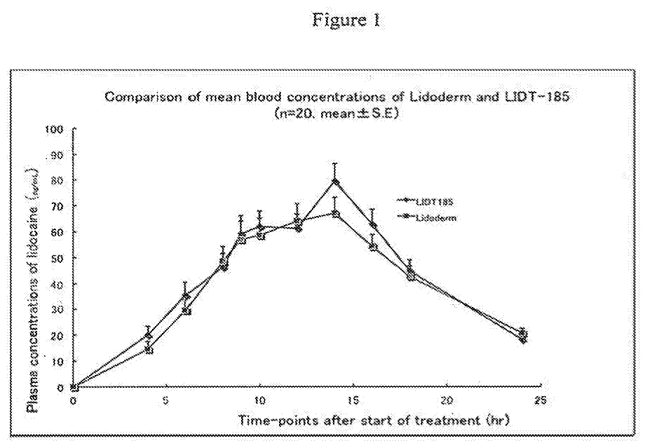

### DETAILED DESCRIPTION OF THE INVENTION

Lidoderm® (lidocaine patch 5%) is comprised of an adhesive material containing 5% lidocaine, which is applied to a non-woven polyester felt backing and covered with a polyethylene terephthalate (PET) film release liner. The release liner is removed prior to application to the skin. The size of the patch is 10 cm×14 cm. Each adhesive patch contains 700 mg of lidocaine (50 mg per gram adhesive) in an aqueous base. It also contains the following inactive ingredients: dihydroxyaluminum aminoacetate, disodium edetate, gelatin, glycerin, kaolin, methylparaben, polyacrylic acid, polyvinyl alcohol, propylene glycol, propylparaben, sodium carboxymethylcellulose, sodium polyacrylate, D-sorbitol, tartaric acid, and urea.

The present invention relates to non-aqueous tapes and patches containing lidocaine and methods of administering these tapes and patches so that patients receive an effective amount of lidocaine without causing undue side effects. The present invention relates to non-aqueous tapes and patches that contain less lidocaine but are bioequivalent to aqueous lidocaine patches. Pharmacokinetics describes, quantitatively, the various steps of drug distribution in the body including the absorption of drugs, distribution of drugs to various organs and the elimination of drugs from the body. Various pharmacokinetic (pK) parameters include maximum observed plasma concentration (Cₘₐₓ), areas under the plasma concentration-time curve (AUCₗₐₛₜ and AUC_{inf}), areas under the first moment curve (AUMCₗₐₛₜ and AUMC_{inf}), time-to-maximum observed plasma concentration (Tₘₐₓ), half-life (T_{1/2}), the apparent terminal elimination rate constant (λ_{z}), and mean transit time (MTT). Cₘₐₓ refers to the maximum concentration that a drug achieves in tested area after the drug has been administered. The Area Under the Curve (AUC) is a plot of concentration of drug in blood plasma against time. The area is computed from the time the drug is administered to the point where concentration in plasma is negligible. The Volume of Distribution (Vd) relates the amount of drug in the body to the measured concentration in the plasma. A large volume of distribution indicates that the drug distributes extensively into body tissues and fluids. Dose proportionality is also a common phrase used pharmacokinetics. Dose proportionality occurs when increases in the administered dose are accompanied by proportional increases in a measure of exposure like AUC or Cₘₐₓ. Thus, an evaluation of dose proportionality usually includes exposure analysis of 3 or more doses to produce a graph. A discussion of various pharmacokinetic parameters and the methods of measuring them can be found in Clinical Pharmacokinetics and Pharmacodynamics: Concepts and Applications, M. Rowland and T. N. Tozer, (Lippincott, Williams & Wilkins, 2010).

Statistical significance may also be measured using Analysis of variance (ANOVA) and the Schuimann's two one-sided t-test procedures at the 5% significance level. For instance, the log-transformed PK exposure parameters Cmax, AUC₀₋₂₄ and AUC_{inf} may be compared to determine statistically significant differences between dosage forms. The 90% confidence interval for the ratio of the geometric means (Test/Reference) may be calculated. In certain embodiments, dosage forms may be said to be “bioequivalent” or “bioequivalence” may be declared if the lower and upper confidence intervals of the log-transformed parameters are within about any of 70-125%, 80%-125%, or 90-125% of one another. A bioequivalent or bioequivalence is preferably declared where the lower and upper confidence intervals of the log-transformed parameters are about 80%-125%.

The non-aqueous tapes and patches of the present invention have a lower amount of lidocaine than comparable aqueous patches. The non-aqueous tapes and patches of the present invention may have lidocaine or its pharmaceutically acceptable salts in amount of from about 0.5 to about 7 wt %, or from about 0.5 to about 6 wt %, or from about 0.5 to about 5 wt %, or from about 0.5 to about 4 wt %, or from about 0.5 to about 3 wt %, or from about 0.5 to about 2.5 wt % or from about 0.5 to about 2 wt % or from about 0.5 to about 1.5 wt % or from about 0.5 to about 1 wt % or from about 1 to about 7 wt %, or from about 1 to about 6 wt %, or from about 1 to about 5 wt %, or from about 1 to about 4 wt %, or from about 1 to about 3 wt %, or from about 1 to about 2.5 wt % or from about 1 to about 2 wt % or from about 1 to about 1.5 wt % or from about 1.5 to about 7 wt %, or from about 1.5 to about 6 wt %, or from about 1.5 to about 5 wt %, or from about 1.5 to about 4 wt %, or from about 1.5 to about 3 wt %, or from about 1.5 to about 2.5 wt % or from about 1.5 to about 2 wt % or from about 0.5 to about 1.8 wt % or from about 1 to about 1.8 wt % or from about 1.8% to about 5.6%. The non-aqueous tapes and patches of the present invention may have lidocaine or its pharmaceutically acceptable salts in amount of 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%. 5.7%, 5.8%, 5.9% and 6.0%. The lidocaine and/or its pharmaceutically acceptable salts may be mixed in a plaster, thereby producing a non-aqueous patch in which the lidocaine is completely dissolved, and which is effective to relieve various muscle pains over a long period of time. The amount of lidocaine and/or its reactant in the plaster is preferably 0.1 to 1 mg/cm².

The non-aqueous patch is required to have a low plaster wt. When the size of one patch is 14×10 cm, the plaster wt may be 0.84 to 2.8 g. Because the lidocaine content of the plaster may be 0.5 to 7 wt %, the amount of lidocaine per patch can be kept as 196 mg or less.

In order to make lidocaine present uniformly and stably in the plaster for effective use, the lidocaine content is set to be 0.5 to 7 wt %. The reason for this is that when the lidocaine content is less than 0.5 wt %, the effect of relieving various muscle pains is low, and the desired effectiveness cannot be achieved. In contrast, when the lidocaine content is more than 7 wt %, a large amount of dissolving agent is required to ensure the release of lidocaine. The adhesion of the patch is thereby reduced, and the physical properties of the patch cannot be maintained, failing to cause the patch to be sufficiently attached to the affected part. Another reason is that the lidocaine content is desired to be low.

According to the present invention, a small amount of lidocaine is efficiently dissolved, and thereby the lidocaine can be released stably and reliably over a long period of time. Particularly, the present invention is focused on a dissolving agent that can efficiently dissolve lidocaine over a long period of time, revealing that a dissolving agent composed of a mixture of an organic acid and a polyalcohol allows continuous and reliable dissolution of lidocaine.

Examples of organic acids include acetic acid, oleic acid, isostearic acid, etc. Examples of polyalcohols include 1,3-butylene glycol, propylene glycol, dipropylene glycol, polyethylene glycol, glycerin, etc.

The most effective proportion of dissolving agent and lidocaine is 0.5 to 5 wt % of dissolving agent relative to 1 wt % of lidocaine. In this proportion, lidocaine can be stably mixed in a dissolved state, increasing the release rate of the lidocaine to the skin, and causing the drug to effectively permeate into the muscle. Here, the reason for this proportion, i.e., 0.5 to 5 wt % of dissolving agent relative to 1 wt % of lidocaine, is as follows. When the amount of dissolving agent is less than 0.5 wt %, lidocaine cannot be stably dissolved and cannot therefore be favorably released. In contrast, when the amount of dissolving agent is more than 5 wt %, the adhesion of the patch decreases, and sufficient attaching power to the skin cannot be achieved.

Although general starting materials for non-aqueous patches can be used for the plaster, the patch can maintain moderate flexibility by using an elastomer as the base. As the elastomer usable as the base, for example, isoprene rubber, polyisobutylene, and styrene isoprene rubber are preferably used. The amount of elastomer is preferably 10 to 50 wt %, and more preferably 20 to 40 wt %, based on 100 wt % of the plaster.

Further, a tackifier resin for increasing adhesive power can be freely added. Usable examples thereof include rosin-based resin, synthetic petroleum resin, terpene resin, phenol resin, alicyclic petroleum resin, and other resins that are generally used in patches.

The non-aqueous tapes and patches of the present invention may have a tackifier resin in amount of from about 5% to about 70 wt %, or from about 5% to about 60 wt %, or from about 5% to about 50 wt %, or from about 5% to about 40 wt %, or from about 5% to about 30 wt %, or from about 5% to about 25 wt % or from about 5% to about 20 wt % or from about 5% to about 15 wt % or from about 5% to about 10 wt % or from about 10 to about 70 wt %, or from about 10 to about 60 wt %, or from about 10 to about 50 wt %, or from about 10 to about 40 wt %, or from about 10 to about 30 wt %, or from about 10 to about 25 wt % or from about 10 to about 20 wt % or from about 10 to about 15 wt % or from about 15 to about 70 wt %, or from about 15 to about 60 wt %, or from about 15 to about 50 wt %, or from about 15 to about 40 wt %, or from about 15 to about 30 wt %, or from about 15 to about 25 wt % or from about 15 to about 20 wt % or from about 20 to about 70 wt % or from about 20 to about 60 wt % or from about 20 to about 50 wt %, or from about 20 to about 40 wt %, or from about 20 to about 30 wt %. The non-aqueous tapes and patches of the present invention may have a tackifier in amount of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, and 49%.

Polybutene or liquid paraffin may be added as a softener, and menthol, camphor, or the like may be added as a skin stimulant. Moreover, anhydrous silicic acid, zinc oxide, or other inorganic substances, zinc stearate, polyvinylpyrrolidone, or the like can be used as a regulator. Furthermore, antioxidants, UV absorbers, preservatives, sequestrants, and other additives that are designed to prevent the degradation of preparations may be used.

The plaster prepared by mixing these starting materials is held by a substrate comprising nonwoven fabric, woven fabric, knitted fabric, film, or a combination thereof, which can be generally used for patches. As a peeling film covering the plaster surface, a film moderately subjected to a mold release treatment is generally used. Since the drug may be adsorbed to the substrate or peeling film, polyester is generally used as their material; however, any materials can be used unless they cause problems.

The wt of the plaster is preferably in the range of 60 to 200 g/m², and more preferably 80 to 180 g/m². When the plaster wt is less than 60 g/m², it is necessary to increase the proportion of lidocaine to the entire plaster, in order to maintain the sufficient efficacy of lidocaine. In this case, however, lidocaine is not sufficiently dissolved and is crystallized; the crystallized lidocaine cannot be efficiently transferred to the skin. Additionally, it is difficult to control the adhesion of the patch, and the plaster is not flexible against the skin and fails to maintain moderate adhesion. In contrast, when the plaster wt is more than 200 g/m², the plaster is so heavy that plaster dripping easily occurs.

The method of producing the non-aqueous patch of the present invention may be a general method that is conventionally used, such as a hot melt method or a solvent method.

### EXAMPLES

### Example 1

### LIDT-185 Formulation

TABLE 1
LIDT-185 Formulation

| Component | Percentage (%) |
|---|---|
| Lidocaine base | 1.80 |
| Polyisobutylene | 5-15% |
| Dibutylhydroxytoluene | 0.1-0.5% |
| Styrene-isoprene-styrene block copolymer | 10-20% |
| Terpene resin | 10-30% |
| Light anhydrous silicic acid | 0.1-1% |
| Liquid paraffin | 40-55% |
| Isostearic acid | 1-3% |
| Dipropylene glycol | 0.1-1% |

Total amount of plaster 60-200 g/m²
Backing tape: non-woven cloth −(0.8±0.2 mm)
Release liner: polyethylene terephthalate−(65-110 μm)

The styrene-isoprene-styrene block copolymer, polyisobutylene, terpene resin, light anhydrous silicic acid, dibutylhydroxytoluene, and liquid paraffin were placed in a dissolution mixer and dissolved under heating at 150° C. A solution separately prepared by mixing the lidocaine, dipropylene glycol, and isostearic acid, followed by dissolution at 80° C., was added thereto, and the mixture was mixed under heating at 140° C. until the mixture became homogeneous, thereby obtaining a plaster solution. The plaster solution was applied to a polyester film. A polyester fabric was pasted to the film and cooled. The resultant was then cut into a rectangle (about 14 cm×10 cm).

### Example 2

### pK Comparison of LIDT-185 and Lidoderm®

LIDT-185 (as in Example 1 containing 1.8% lidocaine) was compared with a reference drug: Lidoderm® (distributed by Endo Pharmaceuticals Inc.) Twenty healthy adult male and female volunteers with normal skin condition were randomized into the two groups (each 10 subjects) according to a 2-treatment, 2-period crossover design with a minimum 7-day washout period (Table 2). To evaluate the bioequivalence between two formulations of lidocaine, i.e. LIDT-185 and Lidoderm®, a pharmacodynamic study was conducted using the plasma concentration of lidocaine applied in human as a measure in accordance with the “Guidelines on Bioequivalence Studies of Generic Products”. Single topical application on skin was conducted for 12 hours. Three patches (420 cm²) were applied on the volunteers' backs for both study and reference drugs. Time points for blood sampling were before application and at 4, 6, 8, 9, 10, 12, 14, 16, 18 and 24 hours after application (amount of blood taken was about 7 mL). Blood was collected into a heparinized blood collecting tube from the forearm when the pulse is regular. The blood collected was centrifuged (4° C., 3000 rpm, 15 minutes) to obtain plasma (about 3 mL), and immediately stored in a frozen state (−20° C. or less). A plasma concentration of lidocaine in the sample was measured with LC/MS/MS method.

TABLE 2
Study Design

| Application | | Washout | |
|---|---|---|---|
| groups | Period 1 | period | Period 2 |
| Group A | Lidoderm ® | A minimum of | LIDT-185 |
| (n = 10) | | 7 days | |
| Group B | LIDT-185 | | Lidoderm ® |
| (n = 10) | | | |

TABLE 3
Plasma concentrations of lidocaine (ng/mL) and pharmacokinetic parameters for Lidoderm

| Subject | | 4 | 6 | 8 | 9 | 10 | 12 | 14 | 16 | 18 | 24 | Tmax | Cmax | AUC_{0-24 h} |
|---|---|---|---|---|---|---|---|---|---|---|---|---|---|---|
| ID code | before | hr | hr | hr | hr | hr | hr | hr | hr | hr | hr | (hr) | (ng/mL) | (ng · hr/mL) |
| S-1 | N.D | N.D | 11.5 | 24.1 | 27.2 | 30.4 | 33.1 | 41.0 | 37.8 | 25.2 | 10.9 | 14 | 41.0 | 489.3 |
| S-2 | N.D | N.D | 15.1 | 19.6 | 22.5 | 25.8 | 32.1 | 41.4 | 35.3 | 24.0 | 15.2 | 14 | 41.4 | 483.0 |
| S-3 | N.D | 29.1 | 37.0 | 46.9 | 56.7 | 64.7 | 68.6 | 74.3 | 53.5 | 39.5 | 24.7 | 14 | 74.3 | 1010.3 |
| S-4 | N.D | 23.2 | 38.5 | 55.2 | 59.4 | 59.6 | 85.6 | 72.2 | 45.4 | 55.9 | 36.3 | 12 | 85.6 | 1117.1 |
| S-5 | N.D | 9.2 | 14.6 | 21.4 | 24.7 | 28.5 | 32.2 | 32.0 | 28.7 | 20.8 | 10.0 | 12 | 32.2 | 455.4 |
| S-6 | N.D | 10.9 | 28.2 | 36.3 | 37.9 | 43.1 | 47.5 | 69.0 | 51.3 | 43.9 | 15.9 | 14 | 69.0 | 805.0 |
| S-7 | N.D | 12.9 | 23.3 | 41.3 | 48.7 | 71.0 | 77.3 | 80.4 | 70.8 | 53.9 | 21.8 | 14 | 80.4 | 1040.5 |
| S-8 | N.D | 11.1 | 12.7 | 33.2 | 41.7 | 40.7 | 43.9 | 35.4 | 29.4 | 33.6 | 18.6 | 12 | 43.9 | 618.9 |
| S-9 | N.D | 13.6 | 17.8 | 39.1 | 53.1 | 47.2 | 51.7 | 62.6 | 45.9 | 32.6 | 17.8 | 14 | 62.6 | 763.2 |
| S-10 | N.D | N.D | 10.7 | 19.1 | 28.1 | 32.0 | 38.6 | 49.2 | 42.3 | 33.9 | 21.1 | 14 | 49.2 | 585.3 |
| S-11 | N.D | 8.6 | 29.4 | 46.5 | 69.3 | 68.9 | 68.4 | 68.1 | 57.9 | 48.5 | 26.1 | 9 | 69.3 | 988.1 |
| S-12 | N.D | 12.7 | 19.5 | 32.9 | 38.5 | 44.1 | 38.2 | 39.0 | 32.1 | 30.0 | 16.3 | 10 | 44.1 | 618.6 |
| S-13 | N.D | 13.3 | 22.7 | 48.4 | 57.9 | 52.5 | 62.3 | 67.8 | 64.3 | 48.0 | 22.8 | 14 | 67.8 | 943.8 |
| S-14 | N.D | 42.2 | 90.5 | 114.3 | 151.5 | 146.6 | 144.6 | 139.6 | 116.6 | 99.3 | 44.7 | 9 | 151.5 | 2183.4 |
| S-15 | N.D | 19.5 | 37.7 | 87.5 | 83.4 | 78.3 | 74.4 | 67.2 | 64.4 | 41.5 | 15.6 | 8 | 87.5 | 1090.8 |
| S-16 | N.D | 16.5 | 32.7 | 61.3 | 68.1 | 72.3 | 70.4 | 70.2 | 67.3 | 56.7 | 25.7 | 10 | 72.3 | 1103.1 |
| S-17 | N.D | N.D | 40.7 | 82.0 | 80.6 | 85.2 | 87.7 | 81.5 | 62.8 | 48.4 | 17.7 | 12 | 87.7 | 1123.5 |
| S-18 | N.D | 11.1 | 17.8 | 32.9 | 35.4 | 33.7 | 33.3 | 42.5 | 36.8 | 25.2 | 12.2 | 14 | 42.5 | 566.8 |
| S-19 | N.D | 12.8 | 24.8 | 40.0 | 49.3 | 54.1 | 87.4 | 103.9 | 74.9 | 47.1 | 22.3 | 14 | 103.9 | 1066.2 |
| S-20 | N.D | 46.8 | 62.8 | 87.9 | 104.1 | 93.4 | 102.9 | 104.1 | 67.4 | 46.4 | 18.6 | 14 | 104.1 | 1432.3 |
| Mean | 0 | 14.7 | 29.4 | 48.5 | 57.1 | 58.6 | 64.0 | 67.1 | 54.3 | 42.7 | 20.7 | 12.4 | 70.5 | 924.2 |
| SE | | 2.9 | 4.3 | 5.8 | 6.9 | 6.4 | 6.5 | 6.0 | 4.7 | 3.9 | 1.9 | | 6.4 | 90.3 |

TABLE 4
Plasma concentrations of lidocaine (ng/mL) and pharmacokinetic parameters for LIDT-185

| Subject | | 4 | 6 | 8 | 9 | 10 | 12 | 14 | 16 | 18 | 24 | Tmax | Cmax | AUC_{0-24 h} |
|---|---|---|---|---|---|---|---|---|---|---|---|---|---|---|
| ID code | before | hr | hr | hr | hr | hr | hr | hr | hr | hr | hr | (hr) | (ng/mL) | (ng · hr/mL) |
| S-1 | N.D | 12.0 | 19.8 | 43.8 | 40.1 | 43.6 | 51.3 | 52.8 | 40.8 | 27.9 | 9.6 | 14 | 52.8 | 677.0 |
| S-2 | N.D | 5.4 | 9.8 | 18.2 | 18.0 | 36.0 | 31.8 | 41.3 | 41.7 | 31.3 | 13.9 | 16 | 41.7 | 531.6 |
| S-3 | N.D | 20.7 | 30.1 | 44.3 | 46.9 | 44.9 | 49.7 | 51.6 | 36.7 | 28.0 | 13.8 | 14 | 51.6 | 732.4 |
| S-4 | N.D | 10.8 | 25.5 | 42.2 | 43.6 | 58.6 | 56.5 | 66.6 | 66.3 | 79.1 | 37.5 | 18 | 79.1 | 1085.9 |
| S-5 | N.D | 9.3 | 16.1 | 24.1 | 22.4 | 37.6 | 37.5 | 50.3 | 22.2 | 24.6 | 9.9 | 14 | 50.3 | 523.2 |
| S-6 | N.D | 16.2 | 22.0 | 48.1 | 49.2 | 66.0 | 49.6 | 98.9 | 45.6 | 39.8 | 17.7 | 14 | 98.9 | 913.5 |
| S-7 | N.D | 26.3 | 47.4 | 73.1 | 74.6 | 76.8 | 96.2 | 126.6 | 100.8 | 68.4 | 30.4 | 14 | 126.6 | 1488.2 |
| S-8 | N.D | 10.1 | 22.0 | 34.5 | 41.2 | 40.4 | 48.9 | 64.3 | 51.7 | 37.4 | 14.0 | 14 | 64.3 | 749.3 |
| S-9 | N.D | 20.0 | 35.2 | 46.8 | 55.9 | 63.4 | 59.6 | 91.7 | 66.7 | 43.1 | 18.2 | 14 | 91.7 | 1014.6 |
| S-10 | N.D | N.D | 12.5 | 29.4 | 38.5 | 36.6 | 48.0 | 72.5 | 55.9 | 44.0 | 14.8 | 14 | 72.5 | 735.7 |
| S-11 | N.D | 23.2 | 50.7 | 19.2 | 68.1 | 60.2 | 59.8 | 80.6 | 68.3 | 44.4 | 19.1 | 14 | 80.6 | 1010.5 |
| S-12 | N.D | 17.4 | 28.5 | 48.7 | 53.4 | 47.4 | 44.3 | 53.5 | 36.9 | 24.8 | 9.1 | 14 | 53.5 | 702.7 |
| S-13 | N.D | 23.8 | 39.9 | 41.9 | 86.9 | 82.9 | 64.8 | 92.8 | 68.0 | 43.5 | 19.1 | 14 | 92.8 | 1107.8 |
| S-14 | N.D | 39.6 | 111.6 | 106.5 | 156.2 | 149.3 | 140.1 | 150.6 | 127.3 | 85.8 | 39.1 | 9 | 156.2 | 2178.4 |
| S-15 | N.D | 26.7 | 51.2 | 62.7 | 76.2 | 79.6 | 82.4 | 96.0 | 70.5 | 42.7 | 12.8 | 14 | 96.0 | 1179.2 |
| S-16 | N.D | 24.0 | 49.9 | 72.4 | 99.9 | 94.0 | 79.7 | 110.0 | 97.6 | 70.1 | 24.8 | 14 | 110.0 | 1450.7 |
| S-17 | N.D | 27.7 | 21.8 | 30.0 | 55.4 | 56.9 | 44.6 | 42.1 | 46.0 | 24.7 | 14.9 | 10 | 56.9 | 721.4 |
| S-18 | N.D | 11.6 | 16.1 | 27.6 | 26.7 | 35.1 | 36.0 | 61.9 | 52.6 | 21.4 | 13.5 | 14 | 61.9 | 616.9 |
| S-19 | N.D | 8.8 | 22.4 | 34.3 | 51.7 | 43.9 | 57.6 | 86.1 | 79.9 | 65.2 | 18.5 | 14 | 86.1 | 1003.7 |
| S-20 | N.D | 67.8 | 71.0 | 79.5 | 74.0 | 80.9 | 82.9 | 100.2 | 78.5 | 47.6 | 12.3 | 14 | 100.2 | 1410.5 |
| Mean | 0 | 20.1 | 35.2 | 46.4 | 59.1 | 61.8 | 61.1 | 79.5 | 62.7 | 44.7 | 18.2 | 13.9 | 81.2 | 991.6 |
| SE | | 3.3 | 5.4 | 5.0 | 7.0 | 6.2 | 5.6 | 6.6 | 5.7 | 4.3 | 2.0 | | 6.5 | 90.7 |

For pharmacokinetic parameters, the significance of sources of variance was examined on analysis of variance via 2-treatment, 2-period cross-over design. To determine bioequivalence, the difference in the mean values of log AUCₜ and Cₘₐₓ between the study and reference drugs as well as a 90% confidence interval of difference in the mean log-transformed value for each parameter were calculated.

TABLE 5
Results from analysis of variance based on the
parameter for bioequivalence determination, i.e.
Cₘₐₓ, transformed to common logarithm
ANOVA table for 2 × 2 crossover design

| Source of | Degree of | Sum of | Mean | | | |
|---|---|---|---|---|---|---|
| variance | freedom | squares | square | F | P-value | |
| Inter- | 19 | 0.8627 | 0.0454 | 6.2775 | 0.000137 | * |
| subject | | | | | | |
| Group or | 1 | 0.0462 | 0.0462 | 1.0186 | 0.326 | |
| carryover | | | | | | |
| effect | | | | | | |
| Subject/ | 18 | 0.8165 | 0.0454 | 6.2714 | 0.000150 | * |
| group | | | | | | |
| Period | 1 | 0.0013 | 0.0013 | 0.1786 | 0.678 | |
| Drug | 1 | 0.0467 | 6.4524 | 6.4524 | 0.0205 | * |
| Residual | 18 | 0.1302 | 0.0072 | | | |
| error | | | | | | |
| Total | 39 | 1.0409 | | | | |

Criterion for 90% confidence interval of the
difference in mean log-transformed value:
log (0.80) − log (1.25)

| | | | | |
|---|---|---|---|---|
| 90% CI | 1.05 | — | 1.30 | NG |

TABLE 6
Results from analysis of variance based on a
parameter for bioequivalence determination, i.e.
AUC_{0-24 h}, transformed to common logarithm
ANOVA table for 2 × 2 crossover design

| Source of | Degree of | Sum of | Mean | | | |
|---|---|---|---|---|---|---|
| variance | freedom | squares | square | F | P-value | |
| Inter- | 19 | 1.0243 | 0.0539 | 11.6639 | 0.00000146 | * |
| subject | | | | | | |
| Group or | 1 | 0.0286 | 0.0286 | 0.5169 | 0.481 | |
| carryover | | | | | | |
| effect | | | | | | |
| Subject/ | 18 | 0.9957 | 0.0553 | 11.9682 | 0.00000132 | * |
| group | | | | | | |
| Period | 1 | 0.0002 | 0.0002 | 0.0501 | 0825 | |
| Drug | 1 | 0.0107 | 0.0107 | 2.3227 | 0145 | |
| Residual | 18 | 0.0832 | 0.0046 | | | |
| error | | | | | | |
| Total | 39 | 1.1184 | | | | |

Criterion for 90% confidence interval of the
difference in mean log-transformed value:
log (0.80) − log (1.25)

| | | | | |
|---|---|---|---|---|
| 90% CI | 0.99 | — | 1.18 | OK |

The plasma concentrations rose rapidly after applying LIDT-185 and Lidoderm®; Tmax of 13.9 and 12.4 hours and Cₘₐₓ of 81.2±6.5 and 70.5±6.4 ng/mL, respectively. AUC₀₋₂₄ₕ was 991.6±90.7 and 924±90.3 ng·hr/mL (Tables 1 and 2). Among the parameters evaluated, the 90% confidence interval of Cₘₐₓ was log (1.05) to log (1.30). Based on the criterion for bioequivalence as defined in the “Guidelines on Bioequivalence Studies for Generic Products”, i.e. “the test and reference drugs were determined to be biologically equivalent when the 90% confidence interval of the difference in mean log Cₘₐₓ is log (0.8) to log (1.25)”, the two drugs were not determined to be bioequivalent (Table 5) since the 90% higher confidence bound for the test drug was slightly above log (1.25).

The 90% confidence interval of AUC₀₋₂₄ₕ was log (0.99) to log (1.18). Based on the criterion for bioequivalence, the two drugs were determined to be bioequivalent (Table 6).

To investigate the significance for the sources of variance, analysis of variance was carried out for the pharmacokinetic parameters calculated. For the all sources of variance except inter-subject, no significant difference was noted. Therefore the study design was considered to have no problem. In addition, since no significant difference was observed between the two drugs (Tables 5 and 6), a larger sample size may allow a higher-accuracy test for bioequivalence.

The application of LIDT-185 and Lidoderm® for 12 hours did not cause any adverse events in both groups, indicating no difference in the safety of the two drugs.

### Example 3

### Overall pK Comparison of LIDT-185 and Lidoderm®

Study volunteers enrolled in the bioequivalence portion (Cohort 1) of the study which included 52 general population subjects ages ≥18 to ≥65, a total of 18 males and 34 females. The majority of these subjects (51.9%) were Caucasian. Two in this group did not complete the study. Also included in Cohort 1 were 4 geriatric subjects ≥65 years (mean 68.5±4.4), 2 males and 2 females of whom 50% were Caucasian and 50% were black. All geriatric subjects completed the study. During the two-way cross-over study, each subject received application of three patches of either lidocaine patch 1.8% or Lidoderm® Patch 5% for a treatment duration of 12 hours. After a 7-day washout period, each subject received the other patch scenario. Plasma, collected at multiple time points (pre-dose through 48-hour post-dose), was analyzed for lidocaine concentrations according to validated analytical methods.

Study endpoints were (1) comparative pharmacokinetics (PK) between the two patches, including a bioequivalence assessment, (2) absolute lidocaine bioavailability for both patches, (3) relative bioavailability for lidocaine patch 1.8%, and (4) safety. Using the established bioequivalence standards, bioequivalence was demonstrated by the test/reference ratio (lidocaine patch 1.8%/Lidoderm® Patch 5%) of the geometric least-square means (LSM). The 90% confidence intervals (CIs) for Cₘₐₓ, AUC₀₋ₜ, and AUC_{0-inf} were within the 80-125% CI acceptance range (i.e., established bioequivalence standard). The results from the analysis of variance (ANOVA) statistical analysis used to determine bioequivalence is reported in Table 7. The mean descriptive values derived from the observed plasma concentrations of lidocaine are also reported in Table 7.

TABLE 7
Summary Mean Lidocaine Pharmacokinetic Parameter Values (Arithmetic Means) for IV Bolus,
Lidocaine Patch 1.8%, and Lidoderm ® Patch 5% for Combined General and Geriatric Subjects¹

| | | | Tₘₐₓ | | | | | Absolute |
|---|---|---|---|---|---|---|---|---|
| | | Cₘₐₓ | (hour)¹ | AUC₀₋ₜ | AUC_{0-inf} | T_{1/2} | kₑ | BA² |
| Treatment | | (ng/mL) | [95% CI | (ng · h/mL) | (ng · h/mL) | (hour) | (hour)³ | (%) |
| Arm | N | [±SD] | range] | [±SD] | [±SD] | [±SD] | [±SD] | [±SD] |
| Lidocaine patch | 56 | 80.45 | 13.95 | 1160.27 | 1207.41 | 5.56 | 0.13 | 87.16 |
| 1.8% | | (±25.53) | (13.59, | (394.46) | (387.62) | (1.67) | (0.03) | (30.33) |
| | | | 14.58) | | | | | |
| Lidoderm ® | 56 | 75.38 | 12.69 | 1121.01 | 1183.39³ | 6.27 | 0.12 | 22.97 |
| Patch 5% | | (29.96) | (12.47, | (453.05) | (437.38) | (1.77) | (0.03) | (10.37) |
| | | | 13.92) | | | | | |
| 0.7 mg/kg | 56 | 1778.39 | 0.13 | 1981.94 | 1998.56 | 2.92 | 0.24 | Not |
| lidocaine IV | | (2555.09) | (0.14, | (1660.51) | (1667.92) | (0.52) | (0.04) | applicable |
| bolus | | | 0.49) | | | | | |

¹Median value.
²Absolute bioavailability (BA) was defined as (D⁽²⁾ × AUC_{0-∞}⁽¹⁾)/(D⁽¹⁾ × AUC_{0-∞}⁽²⁾) with 1 = patch parameters and 2 = intravenous (IV) parameters.
³Two subjects over 65 years old (Subjects 055 and 056) were excluded from calculations for AUC_{0-inf}, T_{1/2}, and bioavailability because they didn't have sufficient data to calculate ke.

The comparative PK results for Cohort 1 showed that the lidocaine plasma concentration profiles for the two patches were nearly superimposable and were comparable across all values with the exception of bioavailability where lidocaine patch 1.8% was at 87% while Lidoderm® Patch 5% was at 23%. This difference is expected given the difference in amount of drug in the respective patches. Because of a lack of sufficient time points for a geriatric subject, Lidoderm® Patch 5% values kₑ, T_{1/2}, AUC_{0-inf}, and the bioavailability data could not be determined for the geriatric subset population. Because lidocaine patch 1.8% is developed to have superior adhesion to Lidoderm® Patch 5%, the study was designed to allow for tape reinforcement of both patches to assure the patches maintained contact with the skin during the study and to assure optimum drug delivery and exposure. Without the reinforcement, the difference in adhesion properties might have resulted in artificially low and variable results for Lidoderm® Patch 5%, which would have compromised the reference listed drug (RLD) in the bioequivalence assessment. The tape used for reinforcement and the reinforcement procedures were selected to assure contact of the patches to the skin, and not to have any properties beyond ensuring the adhesion that might influence the PK results obtained in the trial.

To determine absolute lidocaine bioavailability and apparent doses for the two patches and intravenous (IV) bolus infusion, volunteers were admitted to the study clinic for the randomized, open-label, two-way cross-over study on the day prior to lidocaine exposure and were discharged 24 hours post-dose. Each subject received a single IV bolus infusion, three lidocaine patches 1.8%, and three Lidoderm® Patch 5%, according to randomization and protocol procedures. Serial blood samples were drawn at pre- and post-dose times as prescribed by the protocol for the determination of plasma lidocaine. In order to obtain accurate results, all patches had to remain completely affixed as applied. In the clinic, the corners of the patches were reinforced with 3M paper tape. Patches were monitored for completeness of adhesion throughout the application. If any lifting of the edges was observed, the loosened edge was reinforced with additional tape. The results for the three key PK parameters are presented in Table 8

TABLE 8
Comparison of Tₘₐₓ, T_{1/2}, and Absolute Bioavailability
for Lidocaine Patch 1.8% versus Lidoderm ® Patch
5% for All Study Subjects

| | T_{1/2} (hours) | Tₘₐₓ (hours) | BA (%) |
|---|---|---|---|
| | Mean ± SD | Median (min, max) | Mean ± SD |
| N | 54 | 56 | 54 |
| Lidocaine patch | 5.56 ± 1.67 | 13.95 (13.59, 14.58) | 87.16 ± 30.33 |
| 1.8% | | | |
| Lidoderm ® Patch | 6.27 ± 1.77 | 12.69 (12.47, 13.92) | 22.97 ± 10.37 |
| 5% | | | |
| P | 0.0077¹ | 0.0005¹ | <.0001² |

¹Compared using the Wilcoxon method.
²Analysis of variance of the untransformed data.
BA = bioavailability; SD = standard deviation.

A comparison of T_{1/2} and Tₘₐₓ between the lidocaine patch 1.8% subjects and the Lidoderm® Patch 5% subjects showed statistical differences, with both parameters slightly greater in the lidocaine patch 1.8% group. Absolute bioavailability was significantly higher (statistically) for the lidocaine patch 1.8%, which is expected given the lower amount of drug contained in the patch. Pharmacokinetic by-sex comparison was determined to assess whether there were any notable differences in lidocaine systemic PK values between males and females of Cohort 1, which is the population used to establish bioequivalence between the two products. Females in general had higher lidocaine exposures on average for Cₘₐₓ, AUC₀₋ₜ, and AUC_{0-∞} relative to males for both products. However, no clinically meaningful differences between formulations can be determined when comparing overall systemic lidocaine concentrations across sexes.

Pharmacokinetic by-age comparison was also performed to assess whether there were any notable differences in lidocaine systemic PK values for Cohort 1 subjects ≥65 years (i.e., geriatric population) as compared to the subjects <65 years of age (i.e., general population) after normal patch application (i.e., single-dose three-patch application for 12-hour period). Both formulations were assessed for intrinsic PK differences as they relate to age. The slight differences for PK parameters observed between the age groups were not deemed statistically significant and cannot be translated into any clinical differences in overall safety or efficacy for the geriatric population. The higher intra-subject variability within the geriatric group is likely a function of the small sample size (n=4). Therefore, the results characterize the geriatric PK in general as consistent with the overall general population PK data, without any statistical differences.

### Example 4

### Photo Irritation, Local Tolerability, and Photosensitivity

Two were conducted to determine the potential of lidocaine patch 1.8% versus Lidoderm® Patch 5% to induce a photoallergic skin reaction using a controlled photopatch testing procedure and to cause irritation when topical application to skin was followed by light exposure, respectively. Irritation at both of the compared non-irradiated lidocaine patch application sites was significantly less than at the irradiated lidocaine patch 1.8% site (p=<0.001). There was no statistical difference in irritation between the irradiated lidocaine patch 1.8% sites when compared to the irradiated Lidoderm® Patch 5% sites. There was no statistical difference in irritation between the non-irradiated lidocaine patch 1.8% sites when compared to the non-irradiated Lidoderm® Patch 5% sites. None of the subjects with either patch developed a reaction that required a change in patch location or discontinuation of treatment due to dose-limiting irritation. There was no evidence of photosensitization and no evidence of significant irritation to either product. Irradiation was associated with erythema. The lidocaine patch effectively decreased erythema following irradiation. It made no difference which lidocaine patch product was used. There was no indication of phototoxicity among any of the subjects on either of the patch products in either study.

### Example 5

### Adhesion Performance

The lidocaine patch 1.8% is designed to be bioequivalent to the Lidoderm® Patch 5%, but with less lidocaine and superior adhesive properties. Because these properties are achieved by compounding the drug within the adhesive mixture layered on to the backing material, adhesion performance is a very important property. Adhesion performance was measured 48 hours following application in 41 subjects who, sequentially by randomization, received lidocaine patch 1.8% and the comparator, Lidoderm® Patch 5%, with a 7 day patch-free resting period between products. The adhesion to the skin was scored as follows: 0—greater or equal to 90% adhered; 1—greater or equal to 75% adhered but less than 90% adhered; 2—greater or equal to 50% adhered but less than 75% adhered; 3—greater than 0% adhered but less than 50% adhered; and 4—0% adhered.

TABLE 9
Frequency Counts

| | Lidocaine 1.8% Patch | Lidoderm ® |
|---|---|---|
| 0 | 20 | 8 |
| 1 | 10 | 6 |
| 2 | 4 | 6 |
| 3 | 1 | 11 |
| 4 | 6 | 10 |
| N | 41 | 41 |

TABLE 10
Mean/Standard Deviation/Median/Totals

| | Lidocaine 1.8% Patch | Lidoderm ® |
|---|---|---|
| Mean | 1.1 | 2.2 |
| Standard Dev | 1.4 | 1.5 |
| Median | 1 | 3 |
| Total Score | 45 | 91 |
| N | 41 | 41 |

After 48 hours of adhesion, 48.8% of the lidocaine patch 1.8% remained essentially adhered to the skin as compared with 17.1% of the Lidoderm® Patch 5%. In total, only 17.1% of the lidocaine patch 1.8% had detached from the skin by 50% or more as compared to 51.2% of the Lidoderm® Patch 5%.

Using statistical methods, the adhesion observed for lidocaine patch 1.8% was non-inferior to Lidoderm® Patch 5%. An ad hoc statistical analysis shows that lidocaine patch 1.8% demonstrated better adhesion than Lidoderm® Patch 5% (P<0.0001).

### Example 6

### Dermal Sensitization and Irritation

To evaluate dermal sensitization and irritation, a study was conducted with 218 subjects who received portions of both lidocaine patches every 48-72 hours for 21 days. Following a 10-17 day “no-patch” resting period, a single 48 hour challenge application was applied. Local tolerability was monitored throughout the study to assess dermal sensitization potential and irritability.

No dermal sensitization was associated with either patch. The lidocaine patch 1.8%, however, resulted in more dermal reactions of greater severity than were observed with Lidoderm® Patch 5%. Nevertheless, the lidocaine patch 1.8% reactions were considered to be generally mild, acceptable to subjects, and not clinically significant.

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

Further advantages of the present immunological compositions and adjuvants of the present invention can be achieved by those skilled in the art based upon the embodiments described herein and are thus specifically within the scope of the present invention.

1-28. (canceled)

29. A non-aqueous patch comprising a plaster and a substrate, wherein the plaster comprises
5.4 wt % lidocaine,
5 to 15 wt % polyisobutylene,
0.1 to 0.5 wt % dibutylhydroxytoluene,
10 to 20 wt % styrene-isoprene-styrene block copolymer,
10 to 50 wt % terpene resin,
0.1 to 1 wt % light anhydrous silicic acid,
40 to 55 wt % liquid paraffin,
1 to 3 wt % isostearic acid, and
0.1 to 1 wt % dipropylene glycol.

30. The non-aqueous patch of claim 29, wherein the terpene resin is from about 10-30 wt %.

31. The non-aqueous patch of claim 29, wherein the terpene resin is about 20 wt %.

32. The non-aqueous patch of claim 29, wherein the substrate is a non-woven cloth.

33. The non-aqueous patch of claim 29, further comprising a polyethylene terephthalate release liner.

34. A method for treating pain in a patient in need thereof, comprising administering to the patient a non-aqueous patch comprising a plaster and a substrate, wherein the plaster comprises
5.4 wt % lidocaine,
5 to 15 wt % polyisobutylene,
0.1 to 0.5 wt % dibutylhydroxytoluene,
10 to 20 wt % styrene-isoprene-styrene block copolymer,
10 to 50 wt % terpene resin,
0.1 to 1 wt % light anhydrous silicic acid,
40 to 55 wt % liquid paraffin,
1 to 3 wt % isostearic acid, and
0.1 to 1 wt % dipropylene glycol.

35. The method of claim 34, wherein the terpene resin is from about 10-30 wt %.

36. The method of claim 34, wherein the terpene resin is about 20 wt %.

37. The method of claim 34, wherein the substrate is a non-woven cloth.

38. The method of claim 34, further comprising a polyethylene terephthalate release liner.

39. The method of claim 34, wherein the pain is associated with postherpetic neuralgia or muscle pain.

40. A method for treating pain in a patient in need thereof comprising administering to the patient a non-aqueous patch comprising a plaster and a substrate, wherein the plaster comprises 1.8 wt % lidocaine; and wherein one or more pharmacokinetic parameters of the lidocaine in the patient is within 70% to 125% of that of an aqueous patch containing 5% lidocaine.
